(19) United States
(12) Patent Application Publication
(10) Pub. No.: US 2007/0118062 A1
(43) Pub. Date: May 24, 2007

(54) Surgical binder undergarment system and method

(76) Inventor: Janet Felicia Fleck, Allen, TX (US)

Correspondence Address: KEVIN MARK KLUGHART, 2516 LILLIAN MILLER PARKWAY, SUITE 115, DENTON, TX, 76210-7205, US

(21) Appl. No.: 11/601,976

(22) Filed: Nov. 20, 2006

Related U.S. Application Data
(60) Provisional application No. 60/597,313, filed on Nov. 22, 2005.

(51) Int. Cl. A61L 15/00

(52) U.S. Cl. 602/75

(57) ABSTRACT

A surgical binder undergarment system and method are disclosed which are designed to prevent skin chafing/irritation on patients recovering from abdominal/thoracic/back surgery and who must wear a surgical binder to prevent post-surgical injury to patient muscle and skin tissue because of incisions necessitated by the surgical procedure. The present invention is generally constructed of a seamless tubular fabric optimally comprising a fabric weave of 50% cotton and 50% polyester that is latex free and stretches over the abdomen/thorax of the patient to form a flexible protective low friction interface between the patient's skin and the surrounding surgical binder. Use of the present invention permits the surgical binder to remain clean with a corresponding reduction in incision infections and increase in overall patient comfort during post-operative recovery.

### CROSS REFERENCE TO RELATED APPLICATIONS

Applicant claims benefit pursuant to 35 U.S.C. § 119 and hereby incorporates by reference Provisional Patent Application for “BINDER BUDDY”, Ser. No. 60/597313, filed Nov. 22, 2005.

### PARTIAL WAIVER OF COPYRIGHT

All of the material in this patent application is subject to copyright protection under the copyright laws of the United States and of other countries. As of the first effective filing date of the present application, this material is protected as unpublished material.

However, permission to copy this material is hereby granted to the extent that the copyright owner has no objection to the facsimile reproduction by anyone of the patent documentation or patent disclosure, as it appears in the United States Patent and Trademark Office patent file or records, but otherwise reserves all copyright rights whatsoever.

### STATEMENT REGARDING FEDERALLY SPONSORED RESEARCH OR DEVELOPMENT

Not Applicable

### REFERENCE TO A MICROFICHE APPENDIX

Not Applicable

1. Field of the Invention

The field of the present invention relates to the use of surgical binders which are used to stabilize tissue and muscle in patients undergoing major abdominal and back surgery. This invention generally relates to articles in United States Patent Class 602 (surgery: splint, brace, or bandage) under subclass 75 (compressive elongate wrap—subject matter comprising an elastic elongated sheet or strip which applies a constricting pressure to the body part around which it is wrapped) and subclass 19 (body—subject matter comprising an appliance which is specially adapted to be applied to a person's thorax to treat the vertebrae and the muscles associated therewith so that a more normal orientation of the body components is achieved).

### BACKGROUND/DESCRIPTION OF THE RELATED ART

### Overview (0100)

In a wide variety of abdominal and thoracic surgeries (including but not limited to gastric bypass operations, liposuction (and other cosmetic procedures), “tummy tucks”, caesarian sections, back surgery, and other abdominal/thoracic procedures) the use of surgical binders is dictated. Surgical binders are essentially bindings which wrap around the abdomen/thorax of the patient and prevent surgical sutures/stitches from tearing/ripping and therefore protect the surgical wound while it heals.

Examples of these surgical binders are generally illustrated in FIG. 1 (0100), with (0101) being extracted from U.S. Pat. No. 5,533,963 issued to Robert L. Hall on Jul. 9, 1996 for DRESSING HOLDER and (0102) which has been extracted from U.S. Pat. No. 6,309,369 issued to Gail S. Lebovic on Oct. 30, 2001 for SURGICAL BINDER AND METHODS OF USE. A generic surgical binder used in conjunction with many preferred embodiments of the present invention is illustrated in FIG. 1 (0103), and may incorporate a variety of means by which the binder is secured to the patient. Note that the generic binder (0103) may incorporate braces (not shown) in some applications.

The general structure of surgical binders as illustrated in FIG. 1 (0101, 0102, 0103) require that these structures be capable of providing significant support for the patient's abdomen to promote healing of the surgical wound and prevent failure of the surgical sutures. This patient support also results in significant binding/chafing of the patient's skin, resulting in discomfort for the patient. Additionally, to prevent infection of the surgical wound, the surgical binder must be kept clean. The requirement for somewhat rigid support in the surgical binder typically means that the binder is difficult to clean/wash. This is especially true in circumstances where the surgical binder incorporates a brace (such as is typically used in back surgery recovery).

Currently, the prior art does not address the issues of surgical binder binding/chafing of the patient's skin, and does not provide any methodology to keep the surgical binder clean to prevent infection of the patient's surgical wound.

### OBJECTIVES OF THE INVENTION

One skilled in the art will recognize that the present invention provides significant improvements to the patient as compared to the prior art. Accordingly, the objectives of the present invention are (among others) to circumvent the deficiencies in the prior art. Some of these benefits which may be present in some embodiments include (but are not limited to) the following objectives:

To provide a surgical binder undergarment that reduces patient chafing caused by friction between the surgical binder and the patient's skin.

To provide a surgical binder undergarment which may be easily cleaned to prevent infection of the patient's wound and also keep the surgical binder clean, thus reducing required washing of the surgical binder.

One skilled in the art will recognize that this list of advantages is not exhaustive and may have application to some embodiments of the present invention and not others. While these objectives should not be understood to limit the teachings of the present invention, in general these objectives are achieved in part or in whole by the disclosed invention that is discussed in the following sections. One skilled in the art will no doubt be able to select aspects of the present invention as disclosed to affect any combination of the objectives described above.

### BRIEF SUMMARY OF THE PRESENT INVENTION

The present invention as generally illustrated in FIG. 2 (0200) is targeted as a surgical binder undergarment which has as its primary purpose the reduction of friction between the surgical binder and the patient's skin. The goal of this invention is to increase patient comfort by reducing the occurrence of skin chafing in the patient cause by excess friction/binding between the surgical binder and the patient's skin, as well as keeping the surgical binder clean.

The present invention achieves this goal by incorporating a soft, seamless, tubular fabric between the patient's skin and the surgical binder. This low friction interface promotes patient comfort, increases mobility, and reduces the chance of infection cause by contaminated surgical binders. The present invention also promotes a cleaner interface between the surgical binder and the patient's surgical wound by permitting ease of washing/drying of the surgical binder undergarment as compared to the cleaning requirements for the surgical binder itself.

Structurally, the present invention surgical binder comprises the following:
(a) A stretchable tubular undergarment comprising an upper tubular edge, lower tubular edge, and seamless fabric connecting said upper and lower edges;
wherein
the surgical binder undergarment is dimensioned for placement around the abdomen of a patient;
the surgical binder undergarment acts as a friction lowering interface between the skin of the patient and a surgical binder that covers the surgical binder undergarment; and
the surgical binder undergarment prevents the surgical binder from chafing the skin of the patient by reducing the friction between the surgical binder and the patient's skin.

FIG. 2 (0200) generally illustrates the claimed surgical binder undergarment, and FIG. 3 (0300) and FIG. 4 (0400) generally illustrate the preferred methods of installing the surgical binder undergarment. FIG. 5 (0500) and FIG. 6 (0600) generally illustrate the use of a surgical binder with the claimed undergarment, with FIG. 7 (0700) generally illustrating the use of the undergarment to reduce skin/binder friction at the upper and lower edges of the surgical binder. FIG. 8 (0800) illustrates a general procedure for using the claimed invention to minimize skin/binder friction and chafing with an aim to improving patient comfort and improving patient recovery time.

### BRIEF DESCRIPTION OF THE DRAWINGS

For a fuller understanding of the advantages provided by the invention, reference should be made to the following detailed description together with the accompanying drawings wherein:

FIG. 1 illustrates a prior art embodiments of a surgical binder extracted from U.S. Pat. No. 5,533,963 issued to Robert L. Hall on Jul. 9, 1996 for DRESSING HOLDER (0101), and from U.S. Pat. No. 6,309,369 issued to Gail S. Lebovic on Oct. 30, 2001 for SURGICAL BINDER AND METHODS OF USE (0102), with a generic binder (0103) used in many applications illustrated in its typical configuration;

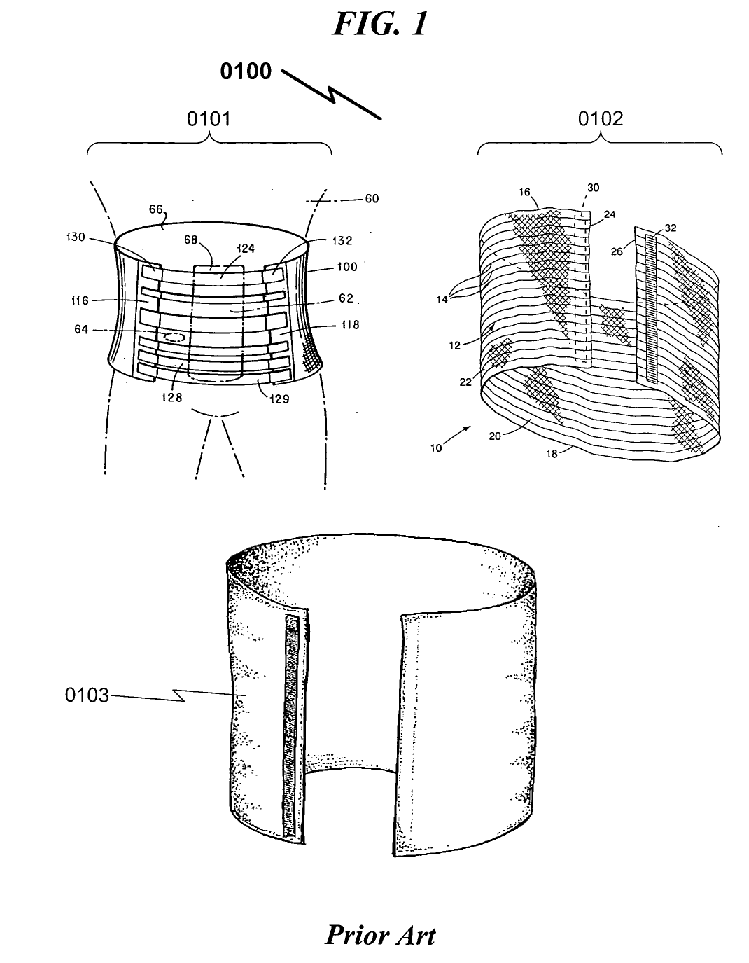

FIG. 2 illustrates a preferred exemplary embodiment of the present invention as installed on a patient prior to application of a surgical binder;

FIG. 3 illustrates a typical upper installation methodology for a preferred exemplary embodiment of the present invention;

FIG. 4 illustrates a typical lower installation methodology for a preferred exemplary embodiment of the present invention;

FIG. 5 illustrates a front patient view of a preferred exemplary embodiment of the present invention as installed on a patient, with subsequent installation of a typical surgical binder;

FIG. 6 illustrates a side patient view of a preferred exemplary embodiment of the present invention as installed on a patient, with subsequent installation of a typical surgical binder;

FIG. 7 illustrates a front patient view of a preferred exemplary embodiment of the present invention as installed on a patient, with subsequent installation of a typical surgical binder, and subsequent overlap of the top and bottom edges of the surgical binder by the top and bottom edges of the present invention surgical binder undergarment;

FIG. 8 illustrates a preferred exemplary method embodiment of a procedure useful in installing/removing some embodiments of the present invention.

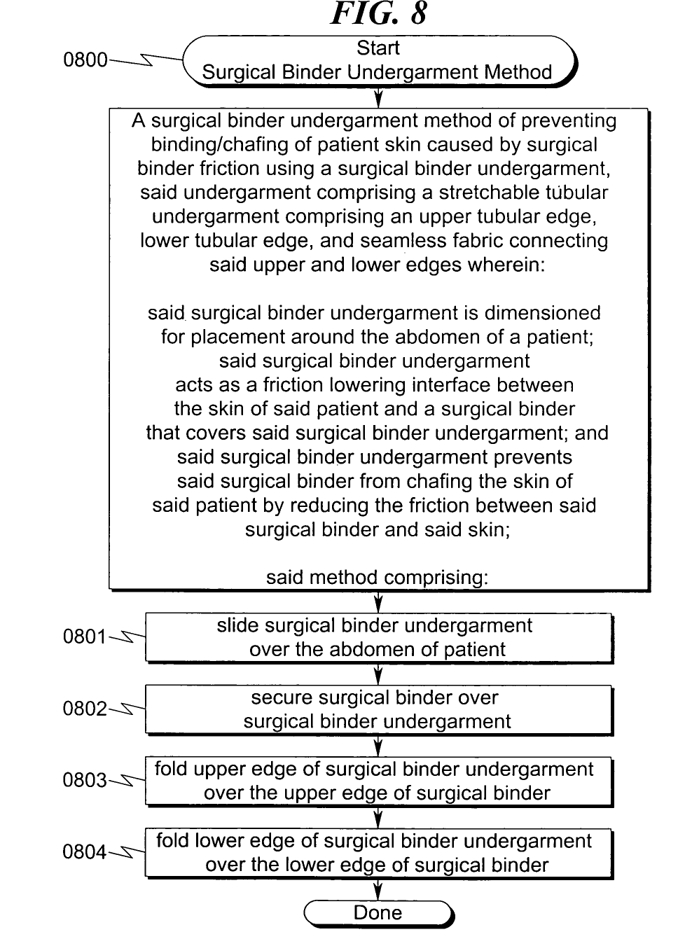

Referring now in detail to the figures wherein like reference numbers like parts throughout, preferred forms of the present invention will now be described.

### DESCRIPTION OF THE PRESENTLY PREFERRED EXEMPLARY EMBODIMENTS

While the present invention is susceptible of embodiment in many different forms, there is shown in the drawings and will herein be described in detailed preferred embodiment of the invention with the understanding that the present disclosure is to be considered as an exemplification of the principles of the invention and is not intended to limit the broad aspect of the invention to the embodiment illustrated.

The numerous innovative teachings of the present application will be described with particular reference to the presently preferred embodiment, wherein these innovative teachings are advantageously applied to the particular problems of a SURGICAL BINDER UNDERGARMENT SYSTEM AND METHOD. However, it should be understood that this embodiment is only one example of the many advantageous uses of the innovative teachings herein. In general, statements made in the specification of the present application do not necessarily limit any of the various claimed inventions. Moreover, some statements may apply to some inventive features but not to others.

### Dimensions Not Limitive

While several preferred embodiments may incorporate preferred dimensions, the present invention scope is not limited by the specific dimensions listed herein.

### Human Application Not Limitive

While many preferred embodiments of the present invention are suitable for use on human beings, there is no limitation to the scope of application of the present invention. Many embodiments of the present invention (with suitable modifications for size) may be equally applied to a wide variety of animal species, including but not limited to horses, livestock, mammals, and other animals which undergo surgical procedures.

### General System Description (0200, 0300, 0400, 0500, 0600)

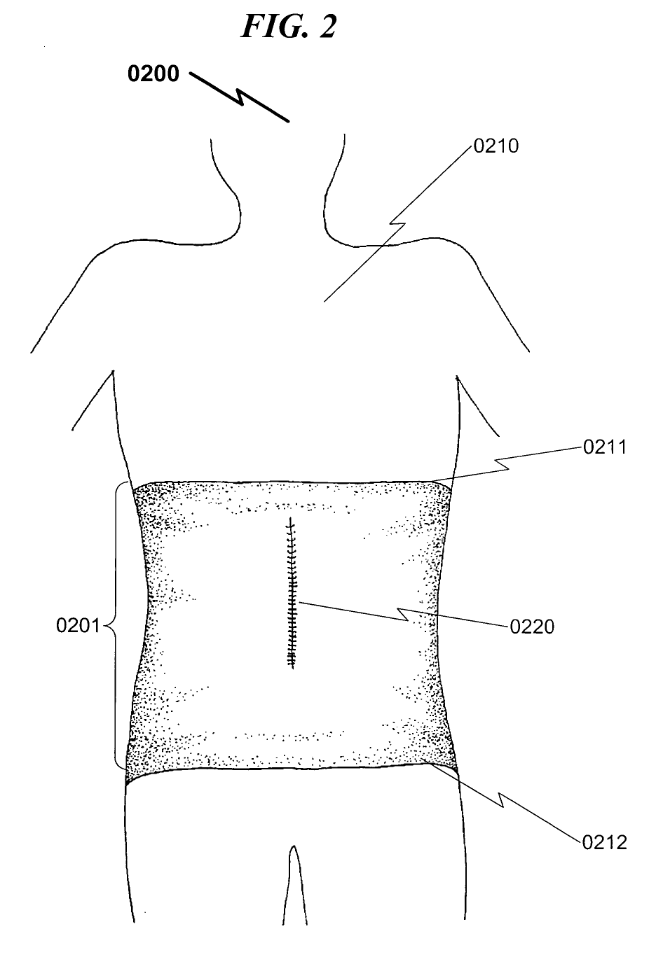

As generally seen in FIG. 2 (0200), the general surgical binder undergarment system comprises a stretchable, tubular, seamless undergarment (0201) having upper (0211) and lower (0212) edges that is dimensioned to form-fit the patient (0210) and cover the patient's wound/scar tissue (0220). Note that illustrations herein do not show any bandages or other would dressings, but one skilled in the art will recognize that these may be present in many applications.

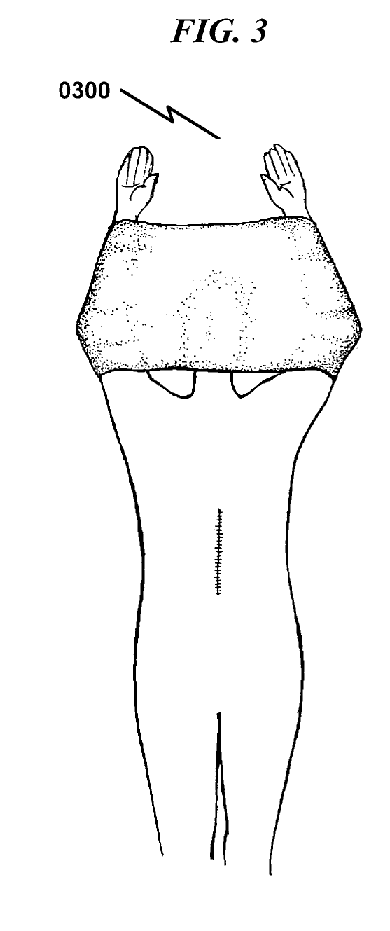

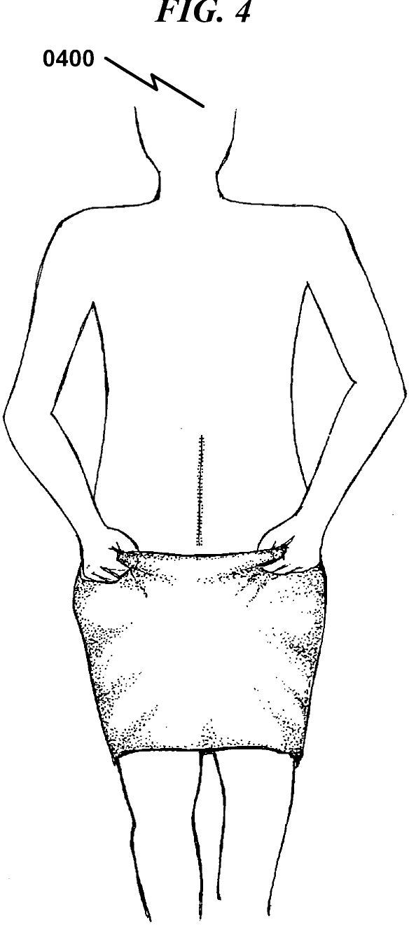

As illustrated in FIG. 3 (0300), this undergarment may be installed from the top of the patient by sliding it over the patient's head and upper torso, or as illustrated in FIG. 4 (0400), installed from the bottom of the patient by sliding it over the patient's legs and lower torso.

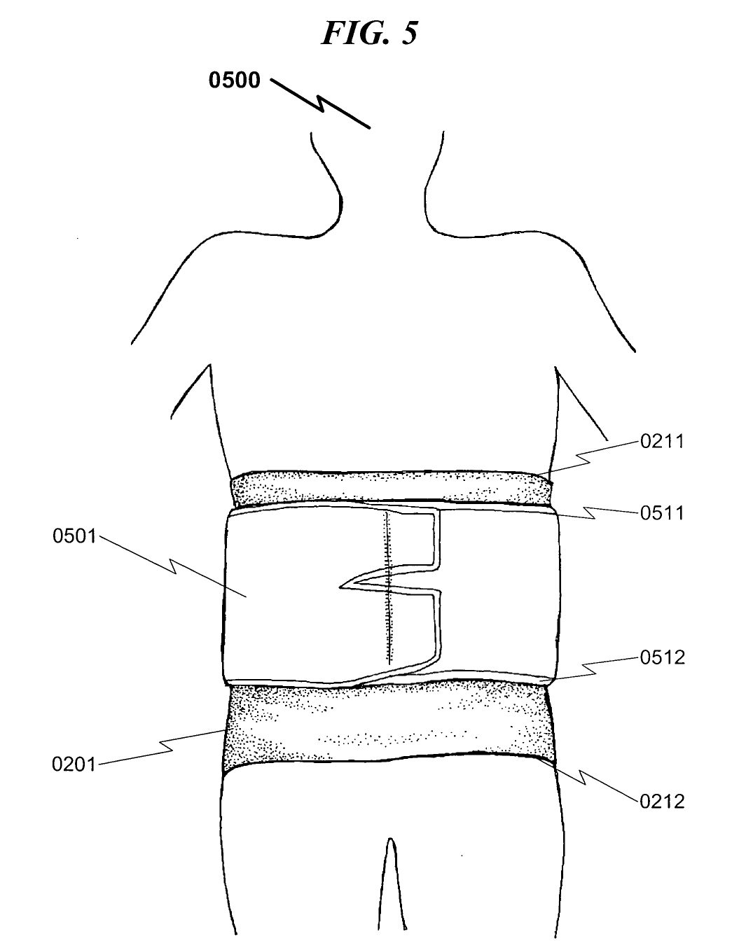

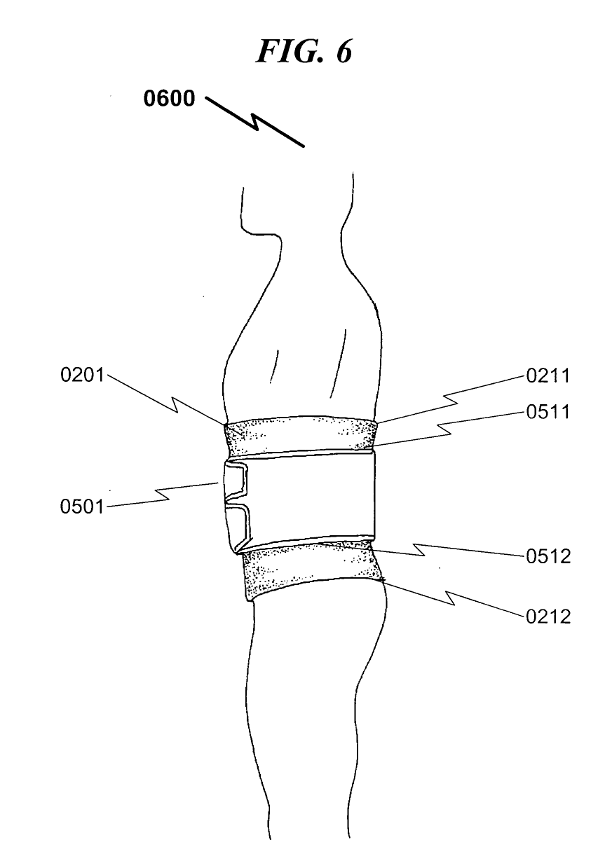

Once properly placed over the patient's abdomen, the surgical binder (0501) may be installed over the surgical binder undergarment (0201) as generally illustrated in FIG. 5 (0500) and FIG. 6 (0600).

### Upper/Lower Surgical Binder Friction Reduction (0700)

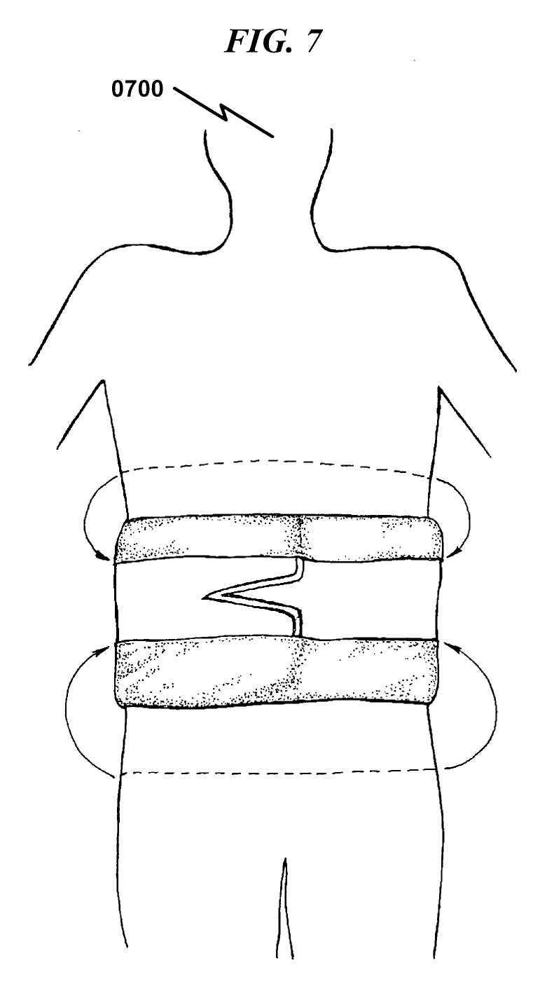

As illustrated in FIG. 5 (0500) and FIG. 6 (0600), many preferred embodiments of the present invention are constructed to incorporate extra undergarment fabric at the upper (0211) and lower (0212) edges to extend beyond the upper (0511) and lower (0512) edges of the surgical binder (0501). This additional fabric overlap may be used to advantage by folding the upper (0211) and/or lower (0212) edges of the surgical binder undergarment over the corresponding upper (0511) and/or lower (0512) edges of the surgical binder as generally illustrated in FIG. 7 (0700). The stretchable nature of the fabric utilized in the present invention is generally sufficient to maintain upper and/or lower edge coverage of the surgical binder as illustrated.

This optional construction/installation of the surgical binder undergarment may in many cases provide significant improvement in patient comfort by eliminating or significantly reducing friction between the patient's skin and the upper (0511) and/or lower (0512) edges of the surgical binder.

### Preferred Exemplary Dimensions

While several preferred embodiments may incorporate a variety of construction dimensions, the present invention may be embodied using several preferred manufacturing dimensions. Generally, the length of the undergarment may be constructed to be approximately 18 inches in many preferred embodiments. This dimension permits the undergarment to extend beyond the upper and lower edges of the surgical binder and also permit “wraparound” of the upper and lower surgical binder edges, to prevent chafing caused by frictional contact between these edges and the patient's skin.

The “width” of the undergarment may be considered its width as laid on a flat surface with the undergarment in its unstretched (preinstalled) state. It has been found through experimentation that undergarment widths of approximately 14 inches will support a size REGULAR for patients with circumferential waist dimensions of 28-48 inches, and undergarment widths of approximately 22 inches will support a size LARGE for patients with circumferential waist dimensions of 44-86 inches. The general rule in many preferred embodiments is to have the unstretched undergarment width to be more than half of the circumferential patient waist dimension.

One skilled in the art will recognize that the undergarment length and width may be varied in a number of ways to accommodate specific patient (and surgical binder) requirements, and that the above guidelines do not limit the teaching scope of the present invention.

### Preferred Exemplary Use/Cleaning

The present invention anticipates the need for the patient to maintain a clean surgical binder to prevent the possibility of infection. While surgical binders can be washed, they often contain a rigid infrastructure, such as stays, embedded plastic supports, and the like. The present invention anticipates that a typical patient will possess two surgical binder undergarments—one which is currently worn under a surgical binder, and the other which is being cleaned after having been previously worn under the surgical binder.

Using this recycling/cleaning procedure, the patient is assured that the surgical binder itself remains clean, and the interface between the patient's healing wound and the surgical binder remains clean and free from potential infections. The selection of fabric materials in the present invention undergarment which are suitable for machine washing/drying promote these patient hygiene goals and encourage the patient to frequently change/clean their surgical binder undergarment.

### Exemplary Preferred Fabric

While many types of fabric are amenable for use in the present invention, many preferred embodiments utilize 20/1 50% polyester yarn and 50% cotton yarn (100% latex free) with a 1×1 rib knit. Manufacturing of the tubular undergarment is optimally accomplished on a circular knitting machine. The fabric is optimally soft and seamless which produces a tubular undergarment that stretches for a perfect fit on the patient.

While a wide variety of fabric thicknesses are possible, optimal sizing permits the undergarment to be worn underneath the patient's support binder or brace following many types of abdominal or back surgeries (including but not limited to weight loss surgery, tummy tuck, liposuction, or back surgery) that require the patient to wear a binder or brace during recovery. While the present invention undergarment is not intended to replace a binder or brace, it is intended to increase the comfort of existing surgical binders/braces for the average six week patient recovery following surgery.

The present invention undergarment's purpose is to create a soft comfortable barrier between the patient's skin and the surgical binder/brace and thereby protect the patient's skin from irritation caused by many binders/braces. The present invention undergarment also plays a very important part in keeping the binder/brace as clean as possible, and this goal is a consideration in the type of fabric used for the present invention undergarment.

### Summary

The present invention system as described herein may be summarized as follows:

A surgical binder undergarment system comprising:
(a) A stretchable tubular undergarment comprising an upper tubular edge, lower tubular edge, and seamless fabric connecting said upper and lower edges;
wherein
said surgical binder undergarment is dimensioned for placement around the abdomen of a patient;
said surgical binder undergarment acts as a friction lowering interface between the skin of said patient and a surgical binder that covers said surgical binder undergarment; and
said surgical binder undergarment prevents said surgical binder from chafing the skin of said patient by reducing the friction between said surgical binder and said skin.

This generalized description may incorporate any of the variations in fabric and structure detailed above. As stated previously, an additional benefit of the present invention in many embodiments is the advantage of keeping the surgical binder clean and preventing post-operative infections caused by soiled surgical binders and/or wound dressings.

### System Variations

The present invention anticipates a wide variety of variations in the basic theme of construction. The examples presented previously do not represent the entire scope of possible usages. They are meant to cite a few of the almost limitless possibilities.

### Generalized Method Embodiment (0800)

The present invention may incorporate a method of using the system as described in an application wherein the invention is used with a surgical binder. This method (0800) may be generally described as follows:
A surgical binder undergarment method of preventing binding/chafing of patient skin caused by surgical binder friction using a surgical binder undergarment, said undergarment comprising a stretchable tubular undergarment comprising an upper tubular edge, lower tubular edge, and seamless fabric connecting said upper and lower edges wherein
said surgical binder undergarment is dimensioned for placement around the abdomen of a patient;
said surgical binder undergarment acts as a friction lowering interface between the skin of said patient and a surgical binder that covers said surgical binder undergarment; and
said surgical binder undergarment prevents said surgical binder from chafing the skin of said patient by reducing the friction between said surgical binder and said skin;
said method comprising:
   (1) sliding said surgical binder undergarment over the abdomen of said patient (0801);
   (2) securing said surgical binder over said surgical binder undergarment (0802);
   (3) folding said upper edge of said surgical binder undergarment over the upper edge of said surgical binder (0803); and
   (4) folding said lower edge of said surgical binder undergarment over the lower edge of said surgical binder (0804).
      This method may be augmented and/or modified according to variations in the surgical binder undergarment system as described above. One skilled in the art will recognize that steps (3) and/or (4) may be optional depending on whether there is a need for friction reduction at the upper and/or lower edges of the surgical binder. Additionally, it will be evident to one skilled in the art that the above steps may be reversed to affect removal of the surgical binder and surgical binder undergarment for undergarment replacement, cleaning, and inspection/cleaning of the patient's wound/scar.

Although a preferred embodiment of the present invention has been illustrated in the accompanying drawings and described in the foregoing Detailed Description, it will be understood that the invention is not limited to the embodiments disclosed, but is capable of numerous rearrangements, modifications, and substitutions without departing from the spirit of the invention as set forth and defined by the following claims.

What is claimed is:

1. A surgical binder undergarment system comprising:
(a) A stretchable tubular undergarment comprising an upper tubular edge, lower tubular edge, and seamless fabric connecting said upper and lower edges;
wherein
said surgical binder undergarment is dimensioned for placement around the abdomen of a patient;
said surgical binder undergarment acts as a friction lowering interface between the skin of said patient and a surgical binder that covers said surgical binder undergarment; and
said surgical binder undergarment prevents said surgical binder from chafing the skin of said patient by reducing the friction between said surgical binder and said skin.

2. The surgical binder undergarment system of claim 1 wherein said fabric is 20/1 50% polyester yarn and 50% cotton yarn with a 1×1 rib knit.

3. The surgical binder undergarment system of claim 1 wherein said fabric is comprised of 50% cotton and 50% polyester.

4. The surgical binder undergarment system of claim 1 wherein said fabric is latex free.

5. The surgical binder undergarment system of claim 1 wherein said undergarment length is approximately 18 inches.

6. The surgical binder undergarment system of claim 1 wherein said undergarment width is approximately 14 inches.

7. The surgical binder undergarment system of claim 1 wherein said undergarment width is approximately 22 inches.

8. The surgical binder undergarment system of claim 1 wherein said undergarment width is more than half the waist dimension of said patient.

9. The surgical binder undergarment system of claim 1 wherein said upper undergarment edge folds over said upper edge of said surgical binder.

10. The surgical binder undergarment system of claim 1 wherein said lower undergarment edge folds over said lower edge of said surgical binder.

11. A surgical binder undergarment method of preventing binding/chafing of patient skin caused by surgical binder friction using a surgical binder undergarment, said undergarment comprising a stretchable tubular undergarment comprising an upper tubular edge, lower tubular edge, and seamless fabric connecting said upper and lower edges wherein:
said surgical binder undergarment is dimensioned for placement around the abdomen of a patient;
said surgical binder undergarment acts as a friction lowering interface between the skin of said patient and a surgical binder that covers said surgical binder undergarment; and
said surgical binder undergarment prevents said surgical binder from chafing the skin of said patient by reducing the friction between said surgical binder and said skin;
said method comprising:
1) sliding said surgical binder undergarment over the abdomen of said patient; and
2) securing said surgical binder over said surgical binder undergarment.

12. The surgical binder undergarment method of claim 11 wherein said fabric is 20/1 50% polyester yarn and 50% cotton yarn with a 1×1 rib knit.

13. The surgical binder undergarment method of claim 11 wherein said fabric is comprised of 50% cotton and 50% polyester.

14. The surgical binder undergarment method of claim 11 wherein said fabric is latex free.

15. The surgical binder undergarment method of claim 11 wherein said undergarment length is approximately 18 inches.

16. The surgical binder undergarment method of claim 11 wherein said undergarment width is approximately 14 inches.

17. The surgical binder undergarment method of claim 11 wherein said undergarment width is approximately 22 inches.

18. The surgical binder undergarment method of claim 11 wherein said undergarment width is more than half the waist dimension of said patient.

19. The surgical binder undergarment method of claim 11 further comprising the step of folding said upper edge of said surgical binder undergarment over the upper edge of said surgical binder.

20. The surgical binder undergarment method of claim 11 further comprising the step of folding said lower edge of said surgical binder undergarment over the lower edge of said surgical binder.
